(19) United States
(12) Patent Application Publication
(10) Pub. No.: US 2023/0349516 A1
(43) Pub. Date: Nov. 2, 2023

(54) ODORANT MASKING SYSTEM

(71) Applicant: Welker, Inc., Sugar Land, TX (US)

(72) Inventors: Mitchell Von Borstel, Houston, TX (US); Wade Williams, Sugar Land, TX (US); Stephen Russell Sams, Sherman, TX (US)

(21) Appl. No.: 18/297,433

(22) Filed: Apr. 7, 2023

Related U.S. Application Data
(60) Provisional application No. 63/363,888, filed on Apr. 29, 2022.

(51) Int. Cl. F17D 3/10; A61L 9/14; C10L 3/00; B01D 53/26; B01D 53/04

(52) U.S. Cl. CPC F17D 3/10; A61L 9/145; C10L 3/006; B01D 53/261; B01D 53/04; A61L 2209/111; A61L 2209/134; A61L 2209/14; B01D 2253/102; B01D 2253/106; B01D 2257/80; B01D 2257/90

(57) ABSTRACT

An odorant masking system is provided that may be used to introduce an odorant masking agent to natural gas released via a vent stack system. When the odorant masking system senses a pressure increase due to a controlled natural gas release (e.g., during a system blowdown), some of the natural gas is diverted through the system, and odorant masking agent is released from a storage tank. The diverted natural gas and the odorant masking agent subsequently may be mixed and introduced to the natural gas being released through the vent stack system. The mixture of diverted gas and odorant masking agent preferably takes the form of a spray that, when introduced to the natural gas at the vent stack, reduces or even neutralizes the foul smell of odorant associated with the controlled release of natural gas.

### CROSS-REFERENCE TO RELATED APPLICATIONS

This Application claims priority to U.S. Provisional Patent Application Ser. No. 63/363,888, filed on Apr. 29, 2022, entitled “ODORANT MASKING SYSTEM,” currently pending, the entire disclosure of which is hereby incorporated herein by reference.

### FIELD OF INVENTION

The present invention relates generally to a system for masking or substantially mitigating the smell intensity of odorant in natural gas that is released intentionally during a controlled natural gas release.

### BACKGROUND OF INVENTION

Natural gas companies frequently vent or otherwise intentionally release natural gas from their pipelines to the atmosphere (e.g., during system blowdowns). They may perform such controlled natural gas releases for a number of reasons including during emergencies or surplus situations.

Natural gas companies typically introduce, via a pipeline, an odorant to natural gas that aims to alert those nearby of unintentional gas leaks. Such odorants are also therefore present when a controlled natural gas release takes place (e.g., during system blowdowns). As such, when a company performs an intentional release and vents its pipeline gas to the atmosphere, the intentionally foul smell of the odorant is also released. Because of the potency of the odorant in natural gas pipelines, it is not uncommon to receive multiple phone calls reporting gas leaks during scheduled blowdowns, despite the gas company alerting all surrounding areas of the blowdown. Under the right weather and/or gas conditions, the smell of odorant can be detected by civilians nearly 100 miles from the site of the natural gas release that occurs during a pipeline blowdown.

Currently, to reduce the impact of the odorant during small blowdowns and releases, an odorant masking agent or odorant neutralizing agent is introduced to the pipeline (e.g., by spraying a pipe by hand). This only works on a small scale, however. When large releases are performed, it is not feasible to apply such an agent. Thus, the only existing “solution” to the potent odors from large natural gas releases is to warn the nearby civilians before natural gas is released.

### SUMMARY OF INVENTION

The odorant masking system hereof aims to mitigate the scent of odorized natural gas when a portion of a natural gas pipeline is being vented to atmosphere, for example through an emergency shut down (ESD) blowdown stack. The masking system includes a tank to contain an odorant masking agent. The tank may be in fluid communication with spray nozzles positioned and located on top of the blowdown stack (or stacks). Aside from the spray nozzles that are installed on the blowdown stacks, all of the equipment for the odorant masking system is preferably contained in an enclosure to protect the equipment from the weather and elements. The enclosure may be positioned and located proximate to the blowdown stack, or the enclosure may be portable (e.g., coupled to a vehicle, a trailer, or a pallet).

During typical operation of the natural gas pipeline, an ESD line coupled to the odorant masking system is at atmospheric pressure. When the pipeline is over-pressured, natural gas is directed through the ESD line and out the ESD blowdown stack into the atmosphere. The odorant masking system is activated when a pressure sense line connected to the ESD line or the natural gas pipeline senses a change in pressure via a pressure switch. In response to the sensed change in pressure, solenoids of the system may open, allowing the odorant masking agent to be released by way of a blanket pressure from the tank. The odorant masking agent may then be directed through a liquid line, out of the liquid outlet of the system, and to the spray nozzles on the ESD blowdown stack. Additionally, a portion of the natural gas from the pipeline may be diverted through a gas conditioning panel and through a gas line to the spray nozzles. At the spray nozzles installed on the ESD blowdown stack, the odorant masking agent from the liquid line and the diverted gas from the gas line are combined or mixed. The mixing of the odorant masking agent and the gas preferably improves the spraying effect when the liquid is pushed out the spray nozzles and into the ESD blowdown stack. The mixture of odorant masking agent and diverted natural gas may be sprayed into the natural gas that is being released into the atmosphere, which helps mitigate the potent smell of natural gas.

The system may be automated in that the system detects the release of gas into the ESD blowdown stack and begins the spray process without the need of a human operator. A controller equipped with the system can record and track information such as when the system is spraying and for how long, the amount of masking agent in the tank, any issue(s) that may be associated with the system, etc. The system may also be equipped with exterior lights on the enclosure that change color or turn on/off to indicate to operators in the area if the system is spraying, if there are any issues with the system, or if there is low liquid in the tank. The system further may include a throttle valve in fluid communication with the liquid outlet such that a discharge rate of the odorant masking agent can be adjusted by operating the throttle valve.

### BRIEF DESCRIPTION OF DRAWINGS

FIG. 1 is a schematic view of an odorant masking system constructed according to the teachings of the present application.

While the disclosure is susceptible to various modifications and alternative forms, a specific embodiment thereof is shown by way of example in the drawings and will herein be described in detail. It should be understood, however, that the drawings and detailed description presented herein are not intended to limit the disclosure to the particular embodiment disclosed, but to the contrary, the intention is to cover all modifications, equivalents, and alternatives falling within the spirit and scope of the present disclosure as defined by the appended claims.

### DETAILED DESCRIPTION

The invention will now be described with reference to the drawing figures, in which like reference numerals refer to like parts throughout. For purposes of clarity in illustrating the characteristics of the present invention, proportional relationships of the elements have not necessarily been maintained in the drawing figures.

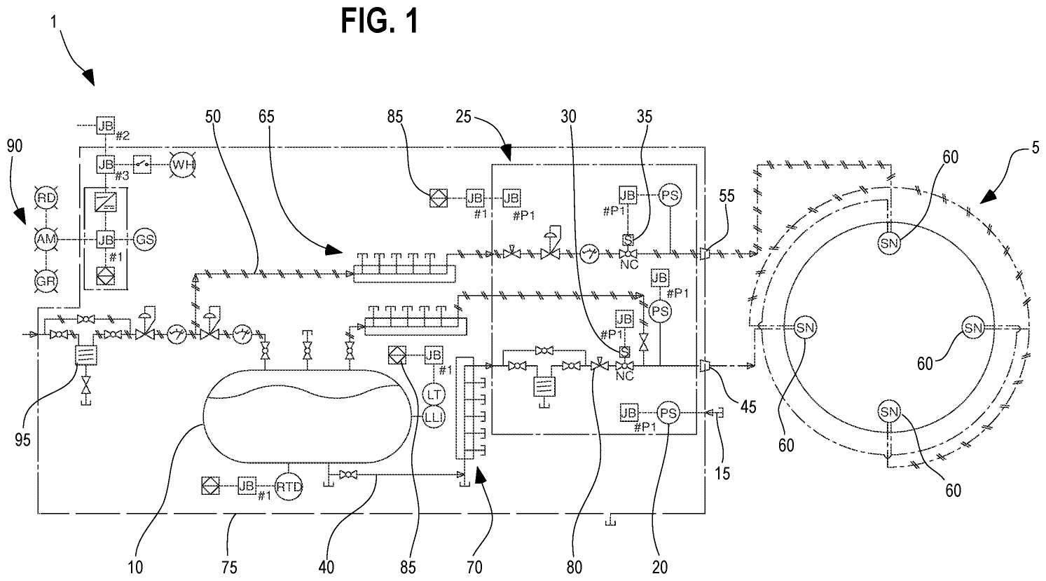

FIG. 1 illustrates an odorant masking system 1 that may be used to introduce an odorant mitigating, neutralizing, or masking agent (not illustrated) to a stream of natural gas (not illustrated) being released by way of a vent stack system 5. The odorant masking agent preferably interacts with the odorant(s) present in the natural gas to mask or blanket the foul-smelling odor of the natural gas being released. The masking agent may be a known or foreseeable solution, such as OdorXice Plus™ odorant removal. Any such masking agent is preferably able to substantially or completely neutralize mercaptans and other organosulfur compounds, including hydrogen sulfide.

The odorant masking agent may be contained and stored in a tank member 10 prior to its introduction into the stream of natural gas. The tank member 10 may be provided in a variety of sizes, but in the illustrated embodiment, the tank member 10 may be sized to contain seventy-five (75) gallons of odorant masking agent.

During standard operation of a natural gas pipeline (not illustrated), an ESD line (not illustrated) is preferably at atmospheric pressure. When a natural gas blowdown occurs, natural gas from the pipeline is not only released from the vent stack system 5, but it is also provided to the ESD line to activate the vent stack system 5 and the odorant masking system 1. For example, a pressure sense line inlet 15 may be in fluid communication with the natural gas pipeline or the ESD line proximate to the vent stack system 5. As a result, when a natural gas blowdown occurs, a pressure switch 20 (and/or sensor) coupled to the pressure sense line inlet 15 may detect a change in pressure in the vent stack system 5.

When the pressure switch 20 detects a change in pressure, a control panel 25 in communication with the pressure switch 20 may activate the odorant masking system 1. For example, the control panel 25 may include various communication methods known and understood in the art for controlling two solenoids, a liquid solenoid 30 and gas solenoid 35 (both contained in the control panel 25). The liquid solenoid 30 may be positioned on a liquid line 40 in fluid communication with the tank member 10 and a liquid outlet 45 of the odorant masking system 1. Additionally, the gas solenoid 35 may be positioned and located on a gas line 50 in fluid communication with the ESD line and a gas outlet 55 of the odorant masking system 1. When the odorant masking system 1 is activated, the control panel 25 may instruct the liquid solenoid 30 and the gas solenoid 35 to open. When the liquid solenoid 30 opens, blanket pressure from the tank member 10 preferably causes the odorant masking agent to be released out of the liquid outlet 45 of the odorant masking system 1. In some embodiments, more or fewer solenoids similar to the solenoids 30, 35 may be provided. Meanwhile, excess natural gas from the ESD line may flow through odorant masking system 1 to the gas outlet 55 via a gas line 50. In alternative embodiments, the natural gas may be additionally pressurized prior to the gas exiting the odorant masking system 1 via the gas outlet 55.

The odorant masking agent from the liquid line 40 and the natural gas from the gas line 50 are preferably mixed and sprayed into the natural gas being released from the vent stack system 5. For example, spray nozzles 60 positioned and located on the vent stack system 5 may receive and mix the odorant masking agent from the liquid line 40 with the gas from the gas line 50. The mixing process preferably improves the spraying effect of the odorant masking agent from the spray nozzles 60 into the vent stack system 5. The spray nozzles 60 preferably direct the mixture of odorant masking agent at the natural gas that is being released from the vent stack system 5. The introduction of the odorant masking agent to the natural gas being released preferably mitigates the odors associated with the natural gas release.

In the illustrated embodiment, the system 1 is further adapted to accommodate additional vent stack systems 5. Five additional connection points 65 are illustrated to which the components used to carry out the release of the natural gas from the gas line 50 may be coupled. Similarly, five additional connection points 70 are illustrated to which the components used to carry out the release of the odorant masking agent may be coupled. As a result, the odorant masking system 1 may introduce odorant masking agent to additional vent stack systems (not illustrated) like the vent stack system 5 illustrated in FIG. 1. In alternative embodiments, the odorant masking system 1 may include more or fewer than five additional connection points 65 and 70 for introducing odorant masking agent and natural gas to additional vent stack systems.

Aside from the spray nozzles 60 that are installed on the vent stack system 5, all of the equipment for the odorant masking system 1 is preferably contained in an enclosure 75 so as to protect the equipment from the weather and elements. The enclosure 75 may be a rectangular housing comprising corrugated steel panels, although in other embodiments, the enclosure 75 may include other suitable structures. In other embodiments, the enclosure 75 may be designed to facilitate transportation of the odorant masking system 1. For example, the odorant masking system 1 may be provided as a trailer-mounted system, a truck-mounted system, or a skid-mounted system in some embodiments. In such embodiments, spray nozzles 60 may be installed on a plurality of vent stack systems 5, and the equipment in the enclosure 75 may be selectively engageable with the spray nozzles 60 on the plurality of vent stack systems 5. For example, the odorant masking system 1 may include flexible hoses (e.g., flexible aluminum or stainless steel pipes) coupled to the liquid outlet 45 and the gas outlet 55. The flexible hoses may assist with selectively engaging the odorant masking system 1 with one or more vent stack systems 5. Accordingly, the odorant masking system 1 may be transported by a truck, a forklift, or another vehicle to be used with various vent stack systems 5.

The odorant masking system 1 also may optionally include various features that help it to carry out its intended functions. For example, the odorant masking system 1 may include a throttle valve 80 in fluid communication with the liquid outlet 45 to selectively control the rate at which odorant masking agent is released from the system 1.

Furthermore, the system 1 may include one or more electronic controllers 85 tasked with reading and tracking the conditions and activity of the system 1. The one or more electronic controllers 85 may be in electrical or wireless communication with one or more exterior lights 90 positioned and located on the enclosure 75. By controlling the operation (e.g., on/off, color, brightness, etc.) of the exterior lights 90, the one or more electronic controllers 85 may inform operators in the area if the system 1 is spraying, if there are any issues with the system 1, and/or if there is low liquid in the tank member 10. In some embodiments, the electronic controllers 85 may control one or both of the solenoids 30, 35.

A conditioning panel 95 may also be provided to condition and clean the pipeline natural gas introduced to the spray nozzles 60 via the gas line 50. The conditioning panel 95 may be provided as a filter and/or a dryer designed to preferably remove aerosols, odorant, moisture, and debris from the natural gas introduced to the system 1. For example, the conditioning panel 95 may include a filter cartridge comprising silica gel, activated charcoal, or a combination thereof. The conditioning panel 95 may be a known or foreseeable solution, such as a F-4 or F-5 filter dryer as manufactured by Welker.

In alternative embodiments, the odorant masking system 1 may further be designed to facilitate cleaning or flushing of the spray nozzles 60. For example, during use of the odorant masking system 1, contaminants (e.g., debris from the environment, residues from the natural gas, or byproducts of the masking agent) may collect in the spray nozzles 60. To remove the contaminants from the spray nozzles 60, the odorant masking system 1 may engage with a pressurized air system, such as an air compressor (not illustrated), through means known in the art. The pressurized air system may generate a flow of air through the spray nozzles 60. Preferably, the flow of air will impart a pressure on the contaminants to dislodge the contaminants from the spray nozzles 60, thereby cleaning the spray nozzles 60. Additionally, in other alternative embodiments, the odorant masking system 1 may receive a solvent instead of or in addition to the pressurize air. The solvent may be a liquid that preferably interacts chemically with contaminants to further facilitate the removal of contaminants from the spray nozzles 60.

As is evident from the foregoing description, certain aspects of the present invention are not limited by the particular details of the examples illustrated herein, and it is therefore contemplated that other modifications, applications, variations, or equivalents thereof, will occur to those skilled in the art. Many such changes, modifications, variations, and other uses and applications of the present constructions will become apparent to those skilled in the art after considering the specification and the accompanying drawings. In addition, unless mention was made above to the contrary, it should be noted that all of the accompanying drawings are not to scale. All such changes, modifications, variations, and other uses and applications which do not depart from the spirit and scope of the present inventions are deemed to be covered by the inventions which are limited only by the claims which follow.

1. An odorant masking system for introducing an odorant masking agent to natural gas released from a natural gas pipeline, the system comprising:
a vent stack system coupled to the natural gas pipeline to release natural gas from the vent stack system;
one or more nozzles positioned and located on or proximate to the vent stack system;
a tank member for containing the odorant masking agent, the tank member fluidly coupled to the one or more nozzles;
a pressure sensor in fluid communication with the vent stack system; and
wherein when the pressure sensor detects a change in pressure, the odorant masking agent from the tank member is supplied to the one or more nozzles.

2. The odorant masking system of claim 1 further comprising a conditioning panel for filtering or drying a portion of the natural gas from the natural gas pipeline, wherein the natural gas that is filtered or dried by the conditioning panel is supplied to the one or more nozzles.

3. The odorant masking system of claim 2, wherein the conditioning panel comprises at least one of silica gel and activated charcoal.

4. The odorant masking system of claim 1 further comprising:
one or more controllers; and
one or more lights in communication with the one or more controllers such that the one or more controllers are configured to activate the one or more lights when the odorant masking agent is provided to the one or more nozzles.

5. The odorant masking system of claim 1, wherein the odorant masking system is selectively engageable with a pressurized air system to generate a flow of air through the one or more nozzles.

6. The odorant masking system of claim 1, wherein the odorant masking system is positioned and located on at least one of a trailer, a pallet, and a truck bed.

7. An odorant masking system for selectively spraying an odorant masking agent, the system comprising:
a vent stack system coupled to a natural gas pipeline to release natural gas from the vent stack system;
a tank member for containing the odorant masking agent;
a liquid line in fluid communication with the tank member;
a gas line for receiving diverted natural gas from the natural gas pipeline;
one or more nozzles in fluid communication with the liquid line and the gas line, the one or more nozzles configured to mix the odorant masking agent from the liquid line and the diverted natural gas from the gas line; and
wherein the one or more nozzles are positioned and located to be directed at the natural gas released from the vent stack system.

8. The odorant masking system of claim 7, further comprising:
a pressure sensor coupled to the vent stack system;
one or more controllers in communication with the pressure sensor;
a liquid solenoid positioned and located on the liquid line;
a gas solenoid positioned and located on the gas line; and
wherein the one or more controllers communicate with the liquid solenoid and the gas solenoid to open at least one of the liquid solenoid and the gas solenoid in response to the pressure sensor detecting a change in pressure.

9. The odorant masking system of claim 7 further comprising a throttle valve in fluid communication with the tank member to control a rate at which the odorant masking agent is released from the tank member.

10. The odorant masking system of claim 7 further comprising a conditioning panel for filtering or drying the diverted natural gas from the gas line.

11. The odorant masking system of claim 7, wherein the odorant masking system is selectively engageable with a pressurized air system to generate a flow of air through the one or more nozzles.

12. The odorant masking system of claim 7, wherein the one or more nozzles are configured to receive a liquid solvent for removing at least one of debris and residue from the one or more nozzles.

13. The odorant masking system of claim 7, further comprising:
one or more lights; and
one or more controllers in communication with the one or more lights, wherein the one or more controllers control a color of the one or more lights to indicate if there are any detected issues with the odorant masking system.

14. An odorant masking system for selectively releasing an odorant masking agent, the system comprising:
a first vent stack system for releasing natural gas from a first natural gas pipeline;
a first set of nozzles positioned and located proximate to the first vent stack system;
a second vent stack system for releasing natural gas from at least one of the first natural gas pipeline and a second natural gas pipeline;
a second set of nozzles positioned and located proximate to the second vent stack system;
a tank member for containing the odorant masking agent; and
wherein the tank member is coupled to at least one of the first set of nozzles and the second set of nozzles to provide the odorant masking agent to at least one of the first vent stack system and the second vent stack system.

15. The odorant masking system of claim 14 further comprising at least one flexible pipe for coupling the odorant masking system to at least one of the first set of nozzles and the second set of nozzles.

16. The odorant masking system of claim 14, wherein the odorant masking system is positioned and located on at least one of a trailer, a pallet, and a truck bed.

17. The odorant masking system of claim 14 further comprising:
one or more controllers;
one or more lights in communication with the one or more controllers; and
wherein the one or more controllers activate the one or more lights when the one or more controllers detect that the volume of odorant masking agent in the tank member is below a threshold volume.

18. The odorant masking system of claim 14 further comprising a pressure sensor in fluid communication with at least one of the first vent stack system and the second vent stack system, wherein when the pressure sensor detects a change in pressure, the odorant masking agent from the tank member is supplied to at least one of the first set of nozzles and the second set of nozzles.

19. The odorant masking system of claim 14 further comprising a throttle valve in fluid communication with the tank member to control a rate at which the odorant masking agent is released from the tank member.

20. The odorant masking system of claim 14 further comprising a conditioning panel for filtering or drying a portion of the natural gas from at least one of the first natural gas pipeline and the second natural gas pipeline, wherein the natural gas that is filtered or dried by the conditioning panel is supplied to at least one of the first set of nozzles and the second set of nozzles.
